(19) United States
(12) Patent Application Publication
(10) Pub. No.: US 2006/0166192 A1
(43) Pub. Date: Jul. 27, 2006

(54) Detection of protease-resistant prion protein following a spontaneous transformation reaction

(75) Inventors: Dieter Gassner, Peissenberg (DE); Ruth Golla, Spatzenhausen (DE)

Correspondence Address: Roche Diagnostics Corporation, Inc., 9115 Hague Road, PO Box 50457, Indianapolis, IN, 46250-0457, US

(73) Assignee: Roche Diagnostics Operations, Inc., Indianapolis, IN (US)

(21) Appl. No.: 11/305,616

(22) Filed: Dec. 16, 2005

Related U.S. Application Data
(63) Continuation of application No. PCT/EP04/06750, filed on Jun. 22, 2004.

(30) Foreign Application Priority Data
Jun. 23, 2003 (DE) DE 10328125.8

(51) Int. Cl. C12Q 1/70; G01N 33/53

(52) U.S. Cl. 435/5; 435/7.1

(57) ABSTRACT

The present invention relates to a method for detecting infectious or pathogenic prion protein with an improved degree of sensitivity. For this, protease-resistant prion protein PrPSc is formed de novo by means of a spontaneous transformation reaction, with nonpathogenic prion protein PrPc in the sample interacting with protease-sensitive low molecular weight PrPSc aggregates to form higher molecular weight protease-resistant PrPSc aggregates.

### RELATED APPLICATIONS

This application is a continuation of PCT/EP2004/006750 filed Jun. 22, 2004, which claims priority to DE 10328125.8 filed Jun. 23, 2003.

### FIELD OF THE INVENTION

The present invention relates to a method for detecting infectious or pathogenic prion protein with an improved degree of sensitivity. For this, protease-resistant prion protein is formed de novo by means of a spontaneous transformation reaction, with nonpathogenic prion protein PrPc in the sample interacting with protease-sensitive low molecular weight PrPSc aggregates to form higher molecular weight protease-resistant PrPSc aggregates.

### BACKGROUND OF THE INVENTION

Prions are the infectious particles which are responsible for transmissible spongiform encephalopathies (TSEs), such as kuru, variant Creutzfeld-Jakob disease (vCJD), spongiform encephalopathy in cattle (BSE), chronic wasting disease (CWD) and scrapie. The main constituent of prions is the glycoprotein PrPSc, which is a conformationally modified isoform of a normal cell surface protein PrPc (Prusiner, PNAS USA 95, 1363-1383, 1998). The disease-associated prion molecule PrPSc is able to replicate by converting normal PrPc prion molecules.

It is assumed that prion replication takes place in accordance with the “nucleation/polymerization” model, which is based on PrPc and PrPSc being in thermodynamic equilibrium in solution (Jarrett and Landsbury, Cell, Vol 73, 1055-1058, 1993; Masel, Jansen and Nowak, Biophys. Chem. 77, 139-152, 1999).

The model is based on the principle that the infectious particle constitutes a multimeric, highly ordered aggregate of PrPSc, while a monomeric PrPSc molecule is unstable and is only stabilized by aggregating with other PrPSc molecules. The rate-determining step of replication is consequently the formation of a nucleus, which then acts to further stabilize PrPSc aggregates.

The PrPSc oligomer is extended at the ends of the aggregate as new PrPc molecules are added on, converted and incorporated. The kinetics of such a “nucleated prion replication” are consequently limited by the number of PrPSc nuclei which are present in the sample and the potential for PrPc and PrPSc to interact with each other.

The prion protein PrPSc has thus far been the only marker available for diagnosing diseases of the TSE type. However, the concentration of PrPSc is so low that, even in the brain, it can only be detected diagnostically in the relatively late phases of a TSE disease. The diagnostic window which exists for detecting TSE diseases is therefore quite restricted.

Efforts are therefore being made to increase the sensitivity of the detection of PrPSc. A method for increasing the sensitivity of the detection of PrPSc in a sample based on the above model of prion replication has recently been developed (Saborio et al., Nature 411, 810-813; 2001 and Soto, Biochem. Soc. Trans. 30, 569-574, 2002, WO 02/04954). This method, which is termed protein misfolding cyclic amplification (PMCA) comprises bringing a sample to be investigated into contact with nonpathogenic PrPc, with small quantities of PrPSc which are present in the sample interacting with added PrPc to form aggregates, the formed aggregates being disaggregated and pathogenic PrPSc being determined in the sample. The method normally consists of several cycles of an experimentally accelerated prion replication. Each cycle consists of two phases. In the first phase, very small quantities of PrPSc interact with a few PrPc molecules and convert them, thereby inducing the growth of PrPSc aggregates.

In the second phase, the administration of ultrasonic waves is used to split these aggregates into small portions, thereby exponentially increasing the number of potential nuclei in each cycle. The cyclic nature of the method at least theoretically allows as many cycles as are required to achieve the amplification status which is desired for detecting PrPSc.

Ultimately, the method is aimed at achieving an exponential increase in the number of template units at the expense of a PrPc substrate with the aid of a cyclic reaction which consists of the phases of aggregation growth and of multiplication of the template units.

The PMCA method, which was applied to a hamster model, has recently also been described for other species, such as mice, sheep, goats, cattle and humans, together with the comment that, depending on the species being used, the strength of the ultrasonication which is to be administered, and which is required for the amplification, clearly has to be adapted, in particular, in dependence on the state of aggregation of the PrPSc polymers concerned (Anderes et al., Poster presentation, Transmissible Spongiform Encephalopathies. New perspectives for prion therapeutics, International Conference, Dec. 1.-3., 2002, Paris, France).

However, the cyclic method for amplifying prions appears to be prone to technical problems and, in the manner described, requires long incubation/sonification cycles.

Tzaban et al. (Biochemistry 41, 12868-12875, 2002) report that previously unknown protease-sensitive PrPSc species are present in prion-infected hamster brains in the form of low molecular weight aggregates. The protease resistance of the aggregates increases as their size increases. However, there is no specific indication that this observation might possibly have diagnostic relevance.

Lucassen et al. (Biochem. 42 (2003), 4127-4135) report that protease-resistant prion protein PrPSc is amplified in vitro by mixing scrapie-infected hamster or mouse brain homogenate with homologous normal brain homogenates.

Horiuchi et al. (PNAS USA, 97 (2000), 5836-5841) investigate binding interactions between heterologous and homologous mouse and hamster PrP isoforms. According to these results, the binding of homologous PrP-sens. to PrP-resist. is not favored as compared with the binding of heterologous PrP-sens.

### SUMMARY OF THE INVENTION

The object underlying the present invention was to provide a simple, rapid and sensitive method for detecting pathogenic prion protein PrPSc in a sample. The achievement of this object, in accordance with the invention, comprises the steps of:

(a) providing a sample to be investigated,

(b) transforming protease-sensitive pathogenic prion protein PrPSc which is endogenously present in the sample by interacting it with nonpathogenic prion protein PrPc to give protease-resistant prion protein PrPSc, without any disaggregation of PrPSc aggregates which have been formed,

(c) incubating with protease, and

(d) determining protease-resistant prion protein PrPSc in the sample.

The method according to the invention is based on the existence of protease-sensitive PrPSc aggregates (Tzaban et al., see above) and their surprising ability to enter into a spontaneous transformation reaction to give protease-resistant PrPSc aggregates.

### BRIEF DESCRIPTION OF THE DRAWINGS

FIG. 1: Various samples were subjected to various treatment steps and investigated.

FIG. 2: Various samples were subjected to various treatment steps and investigated.

FIG. 3: Supernatants from normal brains were mixed with hamster scrapie brain homogenate. Aliquots were subjected to various treatment steps and investigated.

### DESCRIPTION OF THE INVENTION

The invention relates to a method for detecting infectious prion protein with an improved degree of sensitivity. Protease-resistant PrPSc is formed de novo by means of a spontaneous transformation reaction, with endogenous natural PrPc in the sample interacting with protease-sensitive low molecular weight PrPSc aggregates to form higher molecular weight protease-resistant PrPSc aggregates.

In this connection, it is assumed that, in addition to high molecular weight protease-resistant PrPSc aggregates, protease-sensitive low-aggregation PrPSc also exists, in different aggregation states of heterogeneous polymer sizes, in a PrPSc-positive sample. This low-aggregation PrPSc can be used by a PrPc, which is endogenously present in the sample, as a template for a spontaneous transformation reaction which enables protease-resistant PrPSc aggregates to be formed without bringing in any amplification cycles.

Under appropriate conditions, therefore, an increase in the concentration of highly aggregated and protease-resistant PrPSc polymer is brought about by nonpathogenic PrPc which is present in the sample, i.e. PrPc which is endogenously present in the sample and exogenous PrPc which is added, where appropriate, becoming attached to the different low-aggregation and growth-capable PrPSc crystallization nuclei. This in turn leads to protease-resistant PrPSc aggregates being multiplied in number and to a clearly recognizable increase in the sensitivity of PrPSc detection in different detection methods.

The method according to the invention is suitable for detecting prion protein from different organisms such as cattle, mice, hamsters, sheep, goats or humans. It can be used for diagnosing TSE diseases, for example in humans or else in domestic animals, productive animals and wild animals as well.

In its simplest embodiment, the method consists of a step of incubating a sample under membrane-solubilizing conditions in which sphingomyelin/cholesterol-rich detergent-resistant membranes (DRM), i.e. what are termed lipid rafts (Baron and Caughey, Journal Biological Chemistry 2003, Geb 19, e-pub, ahead of print) or caveolae-like domains (CLDs) (Vey et al., Proc. Natl. Acad. Sci. USA, Vol. 93, 14945-14949, 1996) are present and with which PrPc and PrPSc are evidently associated by way of glycosylphosphatidylinositol (GPI) anchors and which promote conversion of PrPc to PrPSc.

Under appropriate conditions, what is termed a spontaneous transformation reaction then takes place by means of PrPc/PrPSc-lipid raft interaction, resulting in a shift of low-aggregation protease-sensitive PrPSc (sPrPSc) to protease-resistant PrPSc superaggregates (rPrPSc) of higher molecular weight.

The advantages of this method are self-evident:
simple to perform,
utilization of endogenous PrPc substrate,
utilization of low-aggregation sPrPSc, which is endogenously present and which was previously lost as a result of protease digestion, and transformation of this sPrPSc into highly aggregated rPrPSc for the purpose of increasing the sensitivity of a conventional detection method
increase in sensitivity in connection with detecting PrPSc in tissues and, for example, cellular blood constituents, such as buffy coat, etc., in which only low concentrations of PrPSc template (in the protease-sensitive, that is sPrPSc, status) exist in association with a potentially high content of PrPc.

Step (a) of the method comprises the provision of a sample to be investigated. The sample can be derived from tissue or body fluids which can contain prion protein, for example brain, nerve tissue or the lymphoreticular system, e.g. blood or blood constituents. The samples are usually prepared in the form of homogenates which contain a lipid raft-conserving detergent, e.g. a nonionic detergent such as Triton X100. Preference is given to not adding ionic detergent, such as SDS, in the case of samples from cattle and humans, in particular. Samples composed of body fluids such as blood, cellular blood constituents, buffy coats, etc., can be prepared by concentrating prion protein-containing cells, e.g. by isolating lymphocytes and other mononuclear cells from anticoagulated whole blood (e.g. Accuspin System Histopaque 1077, Sigma Diagnostics). The sample is preferably prepared under virtually physiological conditions, e.g. pH 6-8 and a salt concentration corresponding to 50-500 mmol of NaCl/l. A protease inhibitor, or a combination of protease inhibitors, e.g. (protease inhibitor cocktail complete, Roche Diagnostics) is advantageously added to the sample in order to inactivate endogenous proteases which are present in the sample. Preference is given to using the sample directly after homogenization or freshly, i.e. without any prior freezing.

Step (b) of the method according to the invention preferably comprises incubating the sample under conditions under which protease-sensitive prion protein PrPSc is spontaneously transformed into protease-resistant prion protein PrPSc. This spontaneous transformation includes nonpathogenic prion protein PrPc becoming attached to the protease-sensitive prion protein PrPSc which is present in the sample.

Where appropriate, exogenous nonpathogenic prion protein PrPc, e.g. PrPc homogenate, which is able to attach itself to low molecular weight PrPSc aggregates which are present in the sample, can be added to the sample for the purpose of further increasing sensitivity. The material which is added to the sample is preferably a fresh homogenate which has not been frozen after the homogenization.

The sample is preferably incubated at a temperature of 20-55° C., in particular of 35-50° C. The incubation takes place for a period of time which is sufficient for achieving an effective increase in sensitivity. The incubation preferably lasts for at least 10 min, e.g. 10-240 min and, particularly preferably, 15-120 min. Where appropriate, a disaggregation step, in which high molecular weight PrPSc aggregates which are originally present in the sample are disaggregated into low molecular weight aggregates, can be carried out prior to step (b). For example, such a step can comprise a single ultrasonic treatment. However, it must be made clear that the method according to the invention is carried out without disaggregating the aggregates which are formed in step (b) and, in particular, without any amplification cycles, i.e., in particular, without several consecutive ultrasonication/incubation cycles.

The protease in accordance with (c) of the method according to the invention is selected such that it is able to cleave nonpathogenic prion protein PrPc, whereas PrPSc, at least from high molecular weight aggregates, is to a large extent resistant to the cleavage. An example of a suitable protease is proteinase K. Particular preference is given to using proteinase K at a concentration of 50-100 μg/ml.

Step (d) of the method according to the invention comprises determining protease-resistant prion protein PrPSc in the sample. This determination can be carried out qualitatively and/or quantitatively using any methods known in the prior art. Examples of suitable methods are immunological methods in which pathogenic prion protein is determined by means of reaction with a specific antibody.

In a particularly preferred embodiment, prion protein is determined by means of western blotting. For this, the sample is fractionated electrophoretically under denaturing conditions, e.g. by means of SDS-PAGE, and the proteins which are contained in the sample are blotted onto a suitable membrane, e.g. a nitrocellulose membrane or polyvinylidene fluoride (PVDF) membrane. The prion protein on the membrane is then visualized by reaction with polyclonal or monoclonal anti-prion antibodies, which can be labeled directly or be detected using a labeled secondary antibody. Preference is given, in this connection, to labeling enzymically and to using a detectable substrate, e.g. a chemiluminescence substrate. Commercially available anti-prion antibodies are specified in the examples.

On the other hand, the determination can also be effected by means of an immunoassay, with the sample being brought, without any prior electrophoretic fractionation, into contact with suitable detection reagents. Preference is given to carrying out the immunoassay as a sandwich assay using a solid phase-side antibody which is directed against the prion protein, e.g. a biotinylated antibody, and a labeled, e.g. an enzyme-labeled, antibody which is directed against the prion protein. Particular preference is given to using a sandwich ELISA for the detection, with use being made of an anti-prion antibody and an enzyme-antibody conjugate as the labeled secondary antibody, together with a detectable enzyme substrate.

The invention will also be explained by means of the following examples.

### Specific Embodiments

### EXAMPLE 1

### Amplifying Bovine PrPSc by Means of a Spontaneous Transformation Reaction

BSE-bovine brain homogenate (20% in 10% sucrose, Ribolyser), which was obtained from the obex region of the medulla oblongata (VLA case 99/00946), was diluted 100-fold with ice-cold:

(a) normal bovine brain homogenate which was obtained from the obex region of the medulla oblongata (10% homogenate in PBS buffer containing protease inhibitor cocktail complete, Roche Diagnostics, +0.5% Triton X100), or

(b) 10% normal hamster brain homogenate (10% homogenate in PBS buffer containing protease inhibitor cocktail complete, Roche Diagnostics, +0.5% Triton X100).

The hamster homogenate (hamster PrPc) was used as a control for comparison with bovine PrPc.

The homogenates were prepared using a Ribolyser appliance in homogenization vessels containing Hybaid ceramic beads. After homogenizing normal brains in PBS buffer containing protease inhibitor cocktail complete, Triton-X100, as previously specified, was added and the samples were centrifuged at 3000 g for 3 min in an Eppendorf centrifuge.

The supernatants from normal brains were placed in an ice bath and, as previously specified, mixed with BSE brain homogenate. 200 μl aliquots were subjected to the following treatment procedures, with 0 min meaning that the samples were kept in the ice bath.

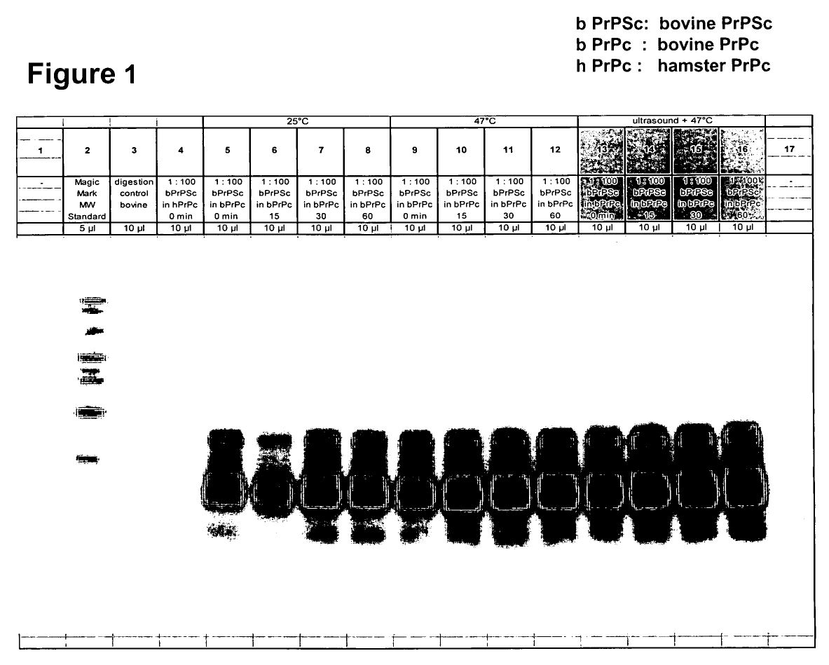

The following samples were investigated in FIG. 1:

Molecular weight markers: FIG. 1, lane 2;

Normal bovine brain (digestion control): FIG. 1, lane 3;

BSE-bovine brain, diluted with normal hamster sample: FIG. 1, lane 4;

BSE-bovine brain, diluted with normal bovine sample:

Incubation at room temperature (25° C.) for 0/15/30/60 min while shaking at 500 rpm (Eppendorf shaker): FIG. 1, lanes 5-8;

incubation at 47° C. for 0/15/30/60 min while shaking at 500 rpm (Eppendorf shaker): FIG. 1, lanes 9-12;

single sonification (1 pulse for 15 sec, intensity 50%) using a microsonicator (Bandelin Electronik, Sono plus, Berlin) fitted with a beaker resonator (BR30) and a sample holding device (EH3), followed by incubation at 47° C. for 0/15/30/60 min while shaking at 500 rpm (Eppendorf shaker): FIG. 1, lanes 13-16.

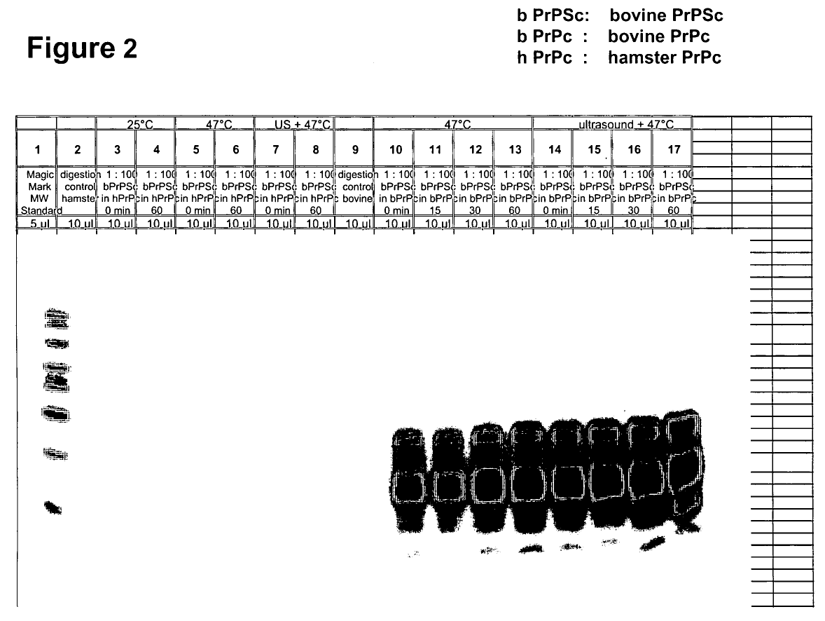

The following samples were investigated in FIG. 2:

Molecular weight markers: FIG. 2, lane 2;

Normal hamster brain (digestion control): FIG. 2, lane 2;

BSE-bovine brain diluted with normal hamster sample:

Incubation at room temperature (25° C.) for 0/60 min while shaking at 500 rpm (Eppendorf shaker): FIG. 2, lanes 3-4;

incubation at 47° C. for 0/60 min while shaking at 500 rpm (Eppendorf shaker): FIG. 2, lanes 5-6;

single sonification (1 pulse for 15 sec, intensity 50%) using a microsonicator (Bandelin Electronik, Sono plus, Berlin) fitted with a beaker resonator (BR30) and a sample holding device (EH3), followed by incubation at 47° C. for 0/60 min while shaking at 500 rpm (Eppendorf shaker): FIG. 2, lanes 7-8;

Normal bovine brain (digestion control): FIG. 2, lane 9;

BSE-bovine brain, diluted with bovine sample:

incubation at 47° C. for 0/15/30/60 min while shaking: FIG. 2, lanes 10-13;

single sonification and incubation at 47° C. for 0/15/30/60 min: FIG. 2, lanes 14-17.

Immediately after that, the samples were digested with proteinase K (100 μg/ml for 60 min at 37° C.). The reaction was stopped by adding the protease inhibitor PMSF to a final concentration of 40 mM. The samples were subjected to an SDS-PAGE, followed by electroblotting onto a PVDF membrane. The membranes were blocked, washed (Dig blocking and washing buffer reagent, Roche Diagnostics) and incubated with the monoclonal anti-prion antibody L42 (R-Biopharm, Darmstadt, 250 ng/ml, 1 h, room temperature); this was then followed by an incubation, for 30 min, with sheep anti-mouse IgG-alkaline phosphatase (40 mU/ml) conjugate Fab fragment (Roche Diagnostics). The reactivity on the membrane was developed by using a CDP-Star chemiluminescence substrate (10 min) followed by visualization using a Lumilmager system (Roche Diagnostics). While the monoclonal anti-prion antibody L42 reacts with human, bovine and sheep PrP, it exhibits what is at best very weak reactivity with hamster PrP in the western blot.

The results depicted in FIGS. 1 and 2 show that a marked improvement in sensitivity was achieved by incubating at 47° C. A further gain in sensitivity could be achieved by sonifying once prior to the incubation.

### EXAMPLE 2

### Amplifying Hamster PrPSc by Means of a Spontaneous Transformation Reaction

Hamster scrapie brains were homogenized in Hank's balanced salt solution using a sterile tissue comminutor to obtain a 10% homogenate; the homogenate was then centrifuged at about 2000 g for 15 min. The supernatant which resulted was stored at −70° C.

10% homogenates of normal hamster brain were prepared in ice-cold PBS buffer, containing protease inhibitor cocktail, Roche Diagnostics, using an Ultraturrax homogenizer. Triton-X100 was added to a final concentration of 0.5% and the samples were centrifuged at 3000 g for 3 min in an Eppendorf centrifuge. A 10% homogenate of bovine brain from the obex region of the medulla oblongata was prepared as a control.

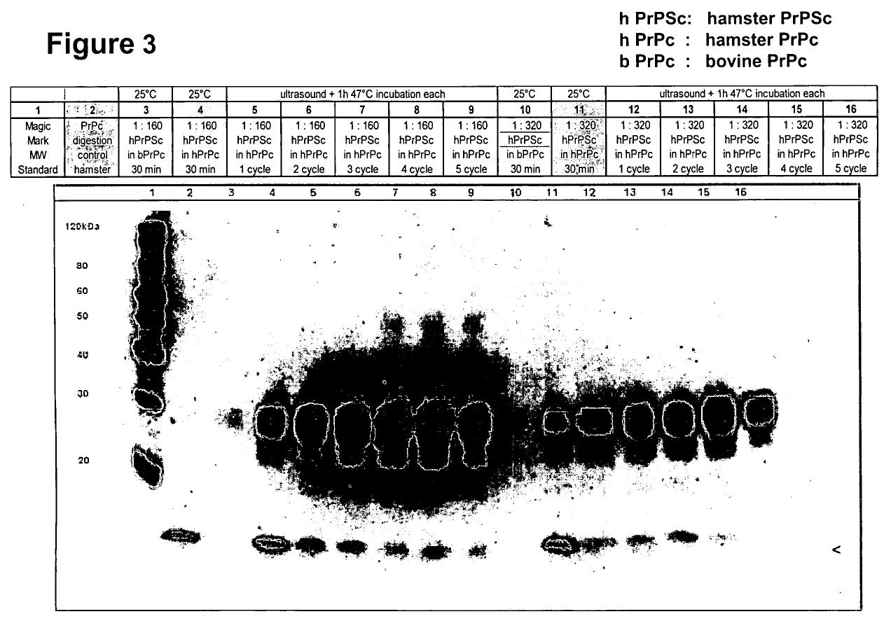

Supernatants from normal brains were mixed with hamster scrapie brain homogenate (1:160, 1:320). 60 μl aliquots were subjected to the following treatment steps and investigated in FIG. 3:

Molecular weight markers: FIG. 3, lane 1

Normal hamster brain (digestion control): FIG. 3, lane 2;

Hamster scrapie brain, diluted:

incubation of hamster PrPSc with hamster PrPc or bovine PrPc as control at room temperature (25° C.) for 30 min while shaking at 500 rpm (Eppendorf shaker): FIG. 3, lanes 3 and 4 (dilution 1:160) and lanes 10 and 11 (dilution 1:320);

single sonification (10 pulses of in each case 0.9 sec with 50% intensity) using a microsonicator (Bandelin Electronik, Sono plus, Berlin) fitted with a beaker resonator (BR30) and a sample holding device (EH3), followed by an incubation at 47° C. for 60 min while shaking at 500 rpm (Eppendorf shaker): FIG. 3, lane 5 (dilution 1:160) and lane 12 (dilution 1:320).

The sonification/incubation cycles were carried out 2-5×: FIG. 3, lanes 6-9 (dilution 1:160) and lanes 13-16 (dilution 1:320).

Immediately after that, the samples were digested with proteinase K (100 μg/ml for 60 min at 37° C.). The reaction was stopped by adding Pefablock up to a final concentration of 10 mM. The samples were subjected to an SDS-PAGE followed by electroblotting onto a PVDF membrane. The membranes were blocked/washed (Dig blocking and washing buffer reagent, Roche Diagnostics) and incubated with the monoclonal anti-prion antibody 3F4 (Signet Laboratories, USA, 500 ng/ml, 1 h, at room temperature) followed by an incubation with sheep anti-mouse IgG-alkaline phosphatase (40 mU/ml) conjugate Fab fragment (Roche Diagnostics) for 30 min. The reactivity on the membrane was developed using the CDP-Star chemiluminescence substrate (15 min) followed by visualization using the Lumilmager system (Roche Diagnostics). While the monoclonal antibody 3F4 reacts with human and hamster PrP, it does not recognize any bovine PrP in the western blot.

The results of the experiment are depicted in FIG. 3. As the arrow in the figure shows, the monoclonal antibody 3F4 reacts with immunoreactive peptide digestion products in the migration front of the SDS gel after the western blot due to the fact that hamster PrPc is still present in excess in lanes 4 and 11. A single ultrasound treatment leads to a significant increase of PrPSc at the expense of PrPc (lanes 5/12; cf. the arrow in the figure). The PMCA method (2-5 cycles) was used for lanes 6-9and 13-16. A decrease in detectable PrPSc can be seen in lanes 9 and 16.

What is claimed is:

1. A method for detecting prion protein PrPSc in a sample, said method comprising the steps of:
(a) providing a sample to be investigated,
(b) producing protease-resistant prion protein PrPSc, de novo, by interacting protease-sensitive pathogenic prion protein PrPSc, which is endogenously present in the sample, with nonpathogenic prion protein PrPc at a temperature of about 20° C. to about 55° C., without a step of disaggregating any of the de novo formed PrPSc aggregates,
(c) incubating the sample with protease, and
(d) determining the presence of protease-resistant prion protein PrPSc in the sample.

2. The method of claim 1 wherein the sample is obtained from cattle, mice, hamsters, sheep, goats or humans.

3. The method claim 2, wherein the sample is derived from brain or nerve tissue.

4. The method claim 2, wherein the sample is derived from fluids isolated from the lymphoreticular system.

5. The method of claim 1 wherein the sample further comprises a detergent.

6. The method of claim 1, wherein the step of producing protease-resistant prion protein PrPSc comprises incubating the sample at a temperature of about 40° C. to about 50° C.

7. The method of claim 6, wherein the incubation lasts for a period of at least 10 min.

8. The method of claim 5, wherein the step of producing protease-resistant prion protein PrPSc comprises incubating the sample at a temperature of about 40° C. to about 50° C. for about 10 to about 120 min.

9. The method of claim 1, wherein the sample is subjected to an ultrasound treatment prior to step (b).

10. The method of claim 8, wherein the sample is subjected to an ultrasound treatment prior to step (b).

11. The method of claim 1, wherein exogenous nonpathogenic prion protein PrPc is added to the sample.

12. The method of claim 10, wherein the protease is proteinase K.

13. The method of claim 12, wherein the proteinase K is used at a concentration of 50-100 μng/ml.

14. The method of claim 1, further comprising the step of quantifying the amount of protease-resistant prion protein PrPSc detected in step (d).

15. The method of claim 1, wherein the determining step is effected using an immunological method.

16. The method of claim 15, wherein the determining step is effected using a western blot.

17. The method of claim 15, wherein the determining step is effected using an immunoassay.

18. The method of claim 17, wherein the immunoassay is an ELISA sandwich assay.

19. The method of claim 1 wherein the method is used for diagnosing TSE diseases in an animal selected from the group consisting of humans, domestic animals, livestock and wild animals.

20. A method for detecting prion protein PrPSc in a sample, said method comprising the steps of:
(a) providing a sample to be investigated,
(b) promoting the de novo formation of protease-resistant prion protein PrPSc in said sample, wherein said promoting step consists of incubating said sample at a temperature of about 20° C. to about 55° C. for about 10 to 240 minutes,
(c) incubating said sample after step (c) with a protease, and
(d) screening the sample after step (d) for the presence of protease-resistant prion protein PrPSc in the sample.

21. The method of claim 20 wherein the sample is the sample is subjected to an ultrasound treatment prior to step (b).
